(19) United States
(12) Patent Application Publication
(10) Pub. No.: US 2018/0235473 A1
(43) Pub. Date: Aug. 23, 2018

(54) SYSTEM, METHOD AND PROCESSOR FOR MONITORING A VITAL SIGN OF A SUBJECT

(71) Applicant: KONINKLIJKE PHILIPS N.V, EINDHOVEN (NL)

(72) Inventors: Mohammed MEFTAH, TILBURG (NL); Willem VERKRUIJSSE, VELDHOVEN (NL); Mukul Julius ROCQUE, EINDHOVEN (NL); Ihor Olehovych KIRENKO, VELDHOVEN (NL)

(21) Appl. No.: 15/753,189

(22) PCT Filed: Aug. 1, 2016
(86) PCT No.: PCT/EP2016/068268

§ 371 Date: Feb. 16, 2018

(30) Foreign Application Priority Data
Sep. 4, 2015 (EP) 15183791.1

(51) Int. Cl. A61B 5/00; A61B 5/024; A61B 5/1455; A61B 5/08

(52) U.S. Cl. CPC A61B 5/0077; A61B 5/6891; A61B 2503/045; A61B 5/14552; A61B 5/0806; A61B 5/02427

(57) ABSTRACT

The present invention relates to a system (10) for monitoring a vital sign of a first subject (12), for example, a neonate, which is in proximity or in contact with a second subject (14), for example, a parent. The system comprises an imaging unit (16) for monitoring the first subject (12) from a distance, to obtain an imaging signal related to a first vital sign of the first subject (12), this imaging unit (16) might be, for example, a camera. The system further comprises a sensing unit (18) for obtaining a sensor signal related to a second vital sign of the second subject (14). The sensing unit (18) is arranged in proximity or at the second subject (14). The first vital sign is of the same type as the second vital sign, thus an analyzing unit (20) can be used for deriving the first vital sign of the first subject (12) from said imaging signal by taking into account said sensor signal.

### FIELD OF THE INVENTION

The present invention relates to a system for monitoring a vital sign of a first subject, which is in proximity or in contact with a second subject. Such situations can occur for example during kangaroo care of a premature baby. The present invention relates further to a processor and a corresponding method as well as a computer program for implementing said monitoring method for use in such a monitoring system.

### BACKGROUND OF THE INVENTION

In the field of medical monitoring it is of high importance to provide a continuous reliable monitoring. This gets even more important the more critical the state of a patient is. Such a case is, for example, the monitoring of a premature baby, a so-called neonate.

Studies such as “Joy E Lawn, Judith Mwansa-Kambafwile, Bernardo L Horta, Fernando C Banos, Simon Cousens, ‘Kangaroo mother care to prevent neonatal deaths due to preterm birth complications’, International Journal of Epidemiology 2010” have shown that kangaroo care can significantly improve the development of a neonate. It is therefore highly recommended to perform kangaroo care during the early development of a neonate.

Kangaroo care means laying a newborn on the chest of a parent. A skin-to-skin contact is desired. Kangaroo care is not only good for the development of the neonate, it also improves parent child bonding.

However, it is also of high importance, to monitor vital signs of a neonate also during kangaroo care. For this purpose usually sensors are attached at the neonate, which can disturb bonding between parent and child, the so called “technology barrier”.

Attaching sensors to the neonate does not only harm bonding between parent and child, it also reduces comfort during kangaroo care. Furthermore, sensors attached to the skin can cause skin irritations or even injuries, especially in case of a neonate.

From EP 2 772 828 A1 an individual body discrimination device and individual body discrimination method are known, wherein a vital sign signal detector detects vital sign signals from captured images of one or more users. A correlation calculator obtains a correlation between the signals derived from different regions of the body of one or more users. An identity determination section then determines whether or not the respective imaged regions of the body belong to the same user based on the correlation between the signal sequences of the vital signs detected from the respective imaged regions of the body. From this document it is also known to provide sensors, which can be worn by a user or which can be included in a controller for a video game, and use vital sign signals derived by the worn sensors in order to improve discrimination of bodies from different users.

### SUMMARY OF THE INVENTION

It is an object of the invention to provide an improved monitoring system for monitoring vital signs of a first subject, which is in proximity or in contact with a second subject, which provides an increased comfort for at least one subject, is easy to handle, delivers reliable vital sign signals, and is preferably applicable for monitoring premature babies during kangaroo care.

According to one aspect of the invention, a system for monitoring a vital sign of a first subject, which is in proximity or in contact with a second subject, is provided, wherein the system comprises:

an imaging unit for monitoring the first subject from a distance, to obtain an imaging signal related to a first vital sign of the first subject,

a sensing unit for obtaining a sensor signal related to a second vital sign of the second subject, said sensing unit being arranged in proximity or at the second subject, wherein the first vital sign is of the same type as the second vital sign, and

an analyzing unit for deriving a first vital sign of the first subject from said imaging signal by taking into account said sensor signal.

According to another aspect of the present invention, a processor is provided for use in a system for monitoring a vital sign of a first subject, which is in proximity or in contact with a second subject, the processor comprising:

a first receiver for receiving an imaging signal related to a vital sign of the first subject,

a second receiver for receiving a sensor signal related to a second vital sign of the second subject, wherein the first vital sign is of the same type as the second vital sign, and

a processing unit for deriving the first vital sign of the first subject from said imaging signal by taking into account said sensor signal.

In yet a further aspect of the present invention, there is provided a corresponding method comprising the steps of:

obtaining an imaging signal related to a first vital sign of the first subject,

obtaining a sensor signal related to a second vital sign of the second subject, wherein said sensor signal is obtained in proximity or at the second subject, wherein the first vital sign is at the same type as the second vital sign, and

deriving the first vital sign of the first subject from said imaging signal by taking into account said sensor signal.

In yet a further aspect of the present invention, there is provided a computer program, which comprises programmed code means for causing a computer to carry out the steps of the method disclosed herein, when said computer program is carried out on the computer.

Preferred embodiments of the invention are defined in the dependent claims. It shall be understood that the claimed method, processor, computer program and medium have similar and/or identical preferred embodiments as the claimed system and as defined in the dependent claims.

It is known to use unobtrusive sensors for monitoring patients. For example, a camera can be used to obtain a photo-plethysmography (PPG) signal. This technique is applicable, for example, for a neonate in a so-called NICU (Neonatal Intensive Care Unit). However, a monitoring system for contactless monitoring of vital signs of a neonate, which is in proximity or in contact with a parent or caregiver without sensors that are arranged at the body of the neonate is currently not available. The present invention is based on the idea to measure an imaging signal from a first subject, which is in proximity or in contact with a second subject and to acquire a vital sign of the second subject via a sensor, which is arranged at or in proximity of the second subject, wherein the acquired vital sign of the second subject is used to determine and/or improve deriving of the vital sign of the first subject from said imaging signal. By acquiring a vital sign of the same type of the second subject with a different sensor, which is in proximity or in contact with the second subject, it is possible to distinguish the first subject from the second subject.

By vital sign of the same type in this context it is meant that if the second sensor obtains, for example, a heart rate signal of the second subject, this heart rate signal is used to derive the heart rate signal of the first subject from said imaging signal, i.e. both times a vital sign of the same type. Further, if the second sensor obtains, for example, a respiratory rate signal of the second subject, this respiratory rate signal is used to derive the respiratory rate signal of the first subject from said imaging signal and if the second sensor obtains, for example, the blood oxygen saturation of the second subject, this blood oxygen saturation is used to derive the blood oxygen saturation of the first subject from said imaging signal.

In accordance with a preferred embodiment, the imaging unit is arranged at a subject support structure or at a predetermined location with respect to the subject support structure for supporting the second subject. Advantageously, the quality of the imaging signal can be improved, as the distance between first subject and imaging unit is constant and therefore fluctuations in the imaging signal arising from movement relative to the imaging unit can be reduced. A subject support structure might be, without limiting the invention to these examples, a chair, a bed, a wheelchair, hospital bed etc. Generally, every structure or furniture that can be used to support, carry or comfort a parent or caregiver during kangaroo care is meant by subject support structure.

In accordance with a further preferred embodiment, the system may comprise the subject support structure, wherein the imaging unit is arranged at a holding structure, which is movably arranged at the subject support structure. This provides an integrated device, which makes the handling very easy, since the cabling and installation is already completed. Furthermore, there is no need for educated personnel to build up the system on site, since the elements of the system may be preassembled. The holding structure might be a movable or flexible arm or an arc, which is movably mounted at the support structure.

In accordance with another preferred embodiment, the system further comprises an actuator for actuating the holding structure and a controller for controlling the actuator based on control signals from a user. This way, the handling can be further improved, since the user can, for example, remotely control, steer and arrange the imaging unit. In cases where, for example, kangaroo care is performed, the parent can stay focused on the child, since no complicated handling or manually arranging the holding structure is necessary.

In accordance with yet a further preferred embodiment, the system comprises a controller for automatically controlling the actuator. This allows increasing the comfort to a very high degree, since the monitoring can be performed without the need of arranging or adjusting. In the case of kangaroo care, the parent can just sit in a kangaroo care chair without the need of assistance from educated personnel and without the need to adjust the holding structure properly. Thus, the parent does not need to take care of anything else but the premature child. Since the effort for adjusting and arranging the system is reduced, also the technological barrier is reduced.

In accordance with another preferred embodiment, the system further comprises an illumination source for illuminating the first subject. The quality of the obtained imaging signal can thus be further improved and the derived vital sign signal can be more reliable. The illumination source can be one or more LED(s), an LED array, a light bulb with or without filter, etc.

In accordance with a further preferred embodiment, the sensing unit is adapted for obtaining one or more of a heart rate signal, respiratory rate signal or blood oxygen saturation signal. These signals can be obtained easily by use of conventional sensors, which are arranged in proximity or at the second subject, such as a pulse oximeter, heart rate monitor, pressure sensor, capacitive sensor, magnetic induction sensor etc. mounted to the subject's finger, wrist, earlobe, chest, etc. Furthermore, these signals are generally the most interesting signals in patient monitoring. Generally, further or other signals may also be used.

In accordance with a further preferred embodiment, the imaging unit is adapted for obtaining PPG signals and the analyzing unit is adapted for deriving the first vital sign of the first subject from said PPG signal by taking into account said sensor signal. In this embodiment, vital signs of the first subject can be derived without attaching sensors at the first subject, wherein the vital signs are reliable, since PPG is a well-known technique.

PPG is an optical measurement technique that evaluates a time-variant change of light reflectance or transmission of an area or volume of interest. PPG is based on the principle that blood absorbs light more than surrounding tissue, so variations in blood volume with every heart beat affect transmission or reflectance correspondingly. Besides information about the heart rate, a PPG waveform can comprise information attributable to further physiological phenomena such as the respiration. By evaluating the transmittance and/or reflectivity at different wavelengths (typically red and infrared), the blood oxygen saturation can be determined.

Conventional pulse oximeters (also called contact PPG device herein) for measuring the heart rate and the (arterial) blood oxygen saturation (also called SpO2) of a subject are attached to the skin of the subject, for instance to a fingertip, earlobe or forehead. Therefore, they are referred to as ‘contact’ PPG devices. A typical pulse oximeter comprises a red LED and an infrared LED as light sources and one photodiode for detecting light that has been transmitted through patient tissue. Commercially available pulse oximeters quickly switch between measurements at a red and an infrared wavelength and thereby measure the transmittance of the same area or volume of tissue at two different wavelengths. This is referred to as time-division-multiplexing. The transmittance over time at each wavelength gives the PPG waveforms for red and infrared wavelengths. Although contact PPG is regarded as a basically non-invasive technique, contact PPG measurement is often experienced as being unpleasant and obtrusive, since the pulse oximeter is directly attached to the subject and any cables limit the freedom to move and might hinder a workflow.

A detailed description of the remote PPG (rPPG) technique may, for example, be found in in Wong M. Y. M. et al.: “Contactless recording of photoplethysmogram on a sleeping bed”, proceedings of the 31st annual international conference of the IEEE engineering in medicine and biology society: engineering the future of biomedicine, EMBC 2009, IEEE, 3 Sep. 2009 (2009-09-03), pages 907-910, which discloses a contactless monitoring method to record a reflective mode photoplethysmogram PPG on a sleeping bed for heart rate (HR) estimation. The electrocardiogram (ECG) and pulse transit time (PTT) were also measured in this study. ECG was measured from subjects' limbs whilst PPG was obtained from their right index fingers and their backs with and without direct contact between the PPG sensor and the subjects' skin respectively. Clear PPG waveforms were obtained from the subjects' backs even though the sensor was not directly attached to their skin. Beat-to-beat HRs derived from the back PPGs were closely correlated with those measured from the finger PPGs and ECGs.

In accordance with a further preferred embodiment, the system comprises a display for displaying vital signs and/or other information of the measurement, wherein the region from which the PPG signal is derived is displayed by the display. A direct feedback of the derived vital signs is thus possible. Furthermore, it can be prevented that vital signs of the wrong subject are monitored, since the region, from which the vital signs are derived, can be directly seen. Thus, a fast adjusting of the imaging unit or holding structure is possible and it is easily possible to correct a measurement in case of a wrong alignment of the imaging unit or holding structure.

In accordance with another preferred embodiment, the system further comprises a projector for projecting light on a region of interest from which the PPG signal is derived. A region of interest can thus be displayed to the user in a simple manner. There is no need to constantly display the region of interest, so that the projector can be switched off after a certain time. The overall system can be kept simpler, since the user may only consider the information of the region of interest only once. Additionally, a constant displaying would again increase the technological barrier. It is further possible to check the region of interest from time to time by projecting the light again on demand or after certain periods of time.

In accordance with yet another embodiment, there is provided a kangaroo care chair for monitoring a vital sign of a first subject, which is in proximity or in contact with a second subject, the chair comprising:

an imaging unit for monitoring the first subject from a distance for obtaining an imaging signal related to a first vital sign of the first subject,

a sensing unit for obtaining a sensor signal related to a second vital sign of the second subject, said sensing unit being arranged at the chair, and wherein the first vital signs is of the same type as the second vital sign, and

a transmitter for transmitting said imaging signal and sensing signal to an analyzing unit for deriving the first vital sign of the first subject from said imaging signal by taking into account said sensor signal.

In a preferred embodiment the chair comprises an arc, which is movably arranged at the chair and wherein the imaging unit is arranged at the arc. This allows that the imaging unit can be integrated and arranged at the chair in a very simple way, without interfering with the design of the chair. Thus, the chair looks less like a medical device and more like furniture and said technological barrier is even smaller in this embodiment.

### BRIEF DESCRIPTION OF THE DRAWINGS

These and other aspects of the invention will be apparent from and elucidated with reference to the embodiment(s) described hereinafter. In the following drawings:

FIG. 1 shows a schematic diagram of an embodiment of a monitoring system according to the present invention,

FIG. 2 shows a diagram exemplarily illustrating three vital sign signals,

FIG. 3 shows a diagram illustrating the method according to the present invention, and

FIG. 4 shows a first embodiment of the monitoring system according to the present invention in form of a kangaroo care chair.

### DETAILED DESCRIPTION OF THE INVENTION

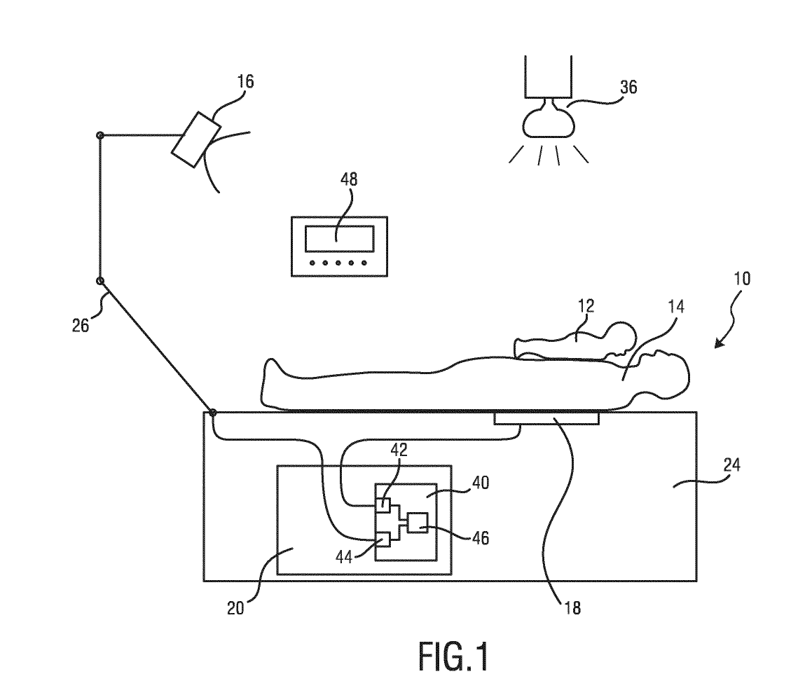

FIG. 1 shows a schematic diagram of an embodiment of a monitoring system 10 according to the present invention. A first subject 12 is lying on a second subject 14, wherein the first subject 12 can be a child or a neonate and the second subject 14 can be a parent or a caregiver.

The system 10 further comprises an imaging unit 16 for obtaining an imaging signal suitable for PPG of the first subject 12. The imaging unit 16 may comprise a camera, a CMOS chip, a CCD chip, a photodiode or any other device known on the art for obtaining an imaging signal.

The system 10 further comprises a sensing unit 18 for obtaining a sensor signal, which is related to a vital sign of the second subject 14. In FIG. 1 this sensing unit 18 is integrated into a support structure 24. However, the sensing unit 18 does not necessarily need to be integrated into the support structure 24 but can also be arranged at the support structure 24 or can be arranged directly at the second subject 14. The sensing unit 18 may comprise one or more sensors like a capacitive ECG/heart rate sensor, a magnetic induction sensor, a pressure sensor or a sound sensor, like for example a microphone.

The system 10 further comprises an analyzing unit 20. This analyzing unit 20 can be an ordinary personal computer, an integrated circuit (IC), a field-programmable gate array (FPGA) an application-specific integrated circuit (ASIC) or anything else that can be used to process signals from said sensing unit 18 and said imaging unit 16.

The system 10 may comprise the subject support structure 24 e.g. a chair, a bed, a hospital bed or the like. The second subject 14 can be supported by said support structure 24. The system 10 further comprises a holding structure 26 for holding said imaging unit 16. The holding structure 26 may be a flexible arm or any structure that can be used to arrange said imaging unit 16 with respect to the first subject 12 and the second subject 14. Although it is shown in FIG. 1, the system 10 does not necessarily comprise said holding structure 26, since the imaging unit 16 does not need to be fixed at the support structure 24, but can be fixed anywhere.

The system 10 may further comprise an illumination unit 36, e.g. an LED array, a light bulb with filter or without filter or any other illumination source that is known in the art.

In the system 10 of FIG. 1 the analyzing unit 20 comprises a processor 40, which comprises a first receiver 42 and a second receiver 44, which receive signals from said sensing unit 18 and said imaging unit 16. The processor 40 further comprises a processing unit 46 for processing the signals received by the first receiver 42 and second receiver 44.

The system 10 of FIG. 1 may further comprise a display 48 for displaying vital signs and/or other information of a measurement.

The system 10 in FIG. 1 works the following way. An imaging signal of the first subject 12 suitable for PPG is obtained by the imaging unit 16 from a certain region of interest of the first subject 12. A sensor signal from the second subject 14 is obtained by said sensing unit 18. The imaging signal and the sensing signal are then transmitted to the analyzing unit 20. Said analyzing unit 20 receives these signals and obtains a vital sign of the first subject 12 by incorporating the sensor signal derived from the second subject 14.

The imaging signal is related to a first vital sign of the first subject 12 and the sensor signal is related to a second vital sign of the second subject 14. The first vital sign and the second vital sign are of the same type. For instance, in an exemplary case the sensing unit 18 is configured to obtain the heart rate of the second subject 14. Then the analyzing unit 20 will obtain the heart rate signal of the first subject 12 from the imaging signal by incorporating the sensor signal. By incorporating the sensor signal the signal quality of the vital sign derived from the imaging signal can be increased. Also influences from a vital sign of the second subject 14 on the imaging signal can be concerned, when deriving a vital sign of the first subject 12. Such influencing can occur, for example, from movement of the first subject 12 due to respiratory movement of the second subject 14. This is advantageous when kangaroo care is performed, since in these cases a skin-to-skin contact between parent and child, i.e. second subject 14 and first subject 12, is desired. Thus, the influence of the vital signs of the parent 14 on the imaging signal derived from the neonate 12 are high.

This technique in principle works with ambient light. Nevertheless, the signal-to-noise ratio (SNR) can be increased, if a light source 36 is used. Advantageous images are obtained, when the light falls perpendicular on the skin of the first subject 12.

The light source 36 could provide visible light to ease aligning the camera 16 and light source 36 but in other cases, e.g. when sleeping is encouraged, provide only near-infrared (NIR) illumination from which PPG signals and blood oxygen saturation may be extracted.

The analyzing unit 20 comprises a processor 40, which comprises a first receiver 42 and a second receiver 44, wherein the first receiver receives the imaging signal related to a first vital sign of the first subject 12 and the second receiver receives the sensor signal related to a second vital sign of the second subject 14. The first vital sign is of the same type as the second vital sign. The processor further comprises a processing unit 46, which derives the first vital sign of the first subject 12 from said imaging signal by taking into account said sensor signal.

To derive a vital sign of the first subject 12, the region of interest from which the imaging signal of the first subject 12 is derived has to be determined and thus the skin from the first subject 12 has to be identified and distinguished from the skin of the second subject 14. To separate the two skin areas, for example a cross-correlation, a matched filter analysis or any alternative correlation technique known in the art could be applied. The sensor signal (e.g. from a capacitive sensor) would provide a high cross-correlation if compared with PPG from skin areas that are of the second subject 14 of whom the second vital sign is being determined with the mentioned sensor signal. If the sensor signal is compared with a vital sign derived from the imaging signal of the first subject 12, this would result in a small cross-correlation.

The cross-correlation is a measure of how well signals conform in phase and frequency. Thus, frequency, phase, phase variations or frequency variations of the first vital sign imaging signal from the first subject 12 would not match/correlate with the second vital sign sensor signal of the second subject 14. Although respiration or SpO2 could in theory be used for this process, pulse variability is the preferred signal since it can relatively easily be measured through non-obtrusive sensors and it also contains better temporal features than respiration or SpO2.

A possible skin separation could be implemented by means of an algorithm, which could comprise breaking up the image in various, non-overlapping, small segments. Each segment is then evaluated for a vital sign (using any known method for rPPG), which is then compared/correlated with the second vital sign of the second subject 14 derived by the sensing unit 18.

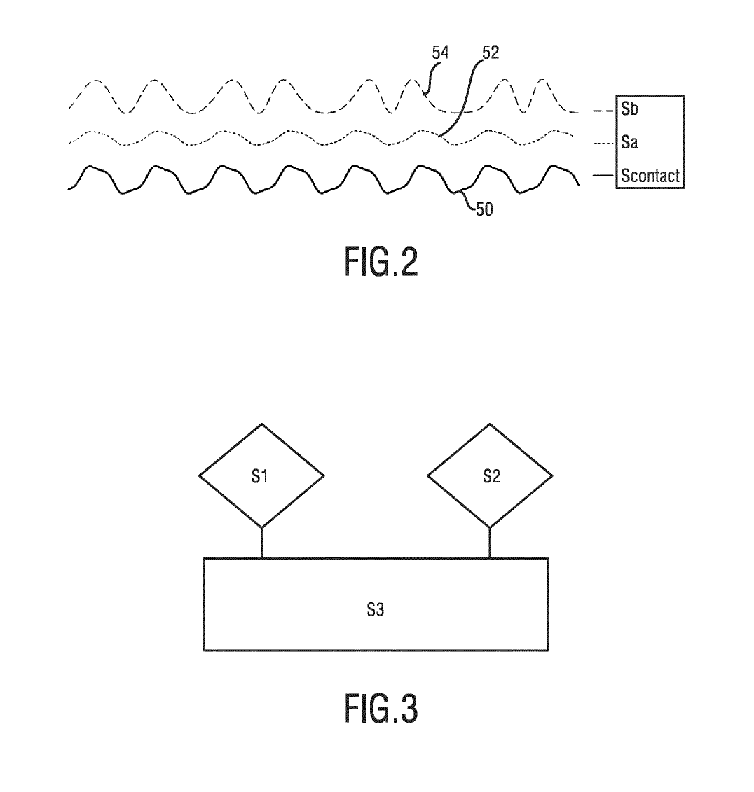

Since the cross-correlation is a measure of how well the signals conform in phase and frequency, the signals have to be corrected for delay in the electronics. This can be implemented by shifting the second vital sign signal derived from the imaging signal and the second vital sign signal derived from the sensor signal with respect to each other until the highest correlation is achieved. This would be some sort of calibration, which can be performed also during a running measurement. Nevertheless, any method for performing a time calibration/time synchronization known in the art may be used. In the signals exemplarily shown in FIG. 2, “Scontact” 50 comprises a vital sign of the second subject 14, measured through a sensing unit 18, for example a finger probe, a capacitive ECG or any other device known for uniquely monitoring the second subject 14. Sa 52 and Sb 54 are vital sign signals originating from rPPG analysis of two regions of interest in an imaging signal. Analyzing pulse rates, i.e. pulses per time interval, results in all three signal segments having the same number of beats/pulses, thus basically the same pulse/heart rate. Consequently, only analyzing the pulse/heart rate cannot be sufficient in order to determine a vital sign of the first subject 12 properly.

However, cross-correlation is a measure for the similarity of two signals. Higher cross-correlation corresponds to more similar signals. Since the second vital sign of the second subject 14, derived by rPPG, and the second vital sign of the second subject 14, derived by the sensing unit 18, are the same vital sign from the same subject, they are supposed to be highly correlated.

The vital sign signals in the example (Scontact, Sa and Sb) are derived from different sensor types, thus the amplitude of the signals is not of interest in the present case. The signals might be normalized before the cross-correlation is calculated. In the present example, the cross-correlation of Scontact with Sa and Sb reveals that Sa is derived from skin of the second subject 14, while Sb is likely from the first subject 12, since the cross-correlation of Scontact and Sa is much closer to 1 (0.99) than the cross-correlation of Scontact and Sb (0.64). These values may vary with signal amplitude.

Anyway, there may be provided a threshold value for the SNR, below which signals will be rejected; or, vice versa, accepted only when the signal's SNR is higher than that a certain threshold.

The system 10 may comprise a patient monitor, e.g. a display 48, where the derived imaging signal and/or sensor signal and/or a derived vital sign of the first subject 12 and/or the derived vital sign of the second subject 14 and/or the region of the first subject 12 from which the imaging signal is recorded might be displayed. Thus, the measurement can be comfortably controlled by the second subject 14 or any other third person. Furthermore, preventing that the imaging signal is obtained from the wrong person/region is eased.

An arrangement of indicator lights and/or the display 48, displaying the live imaging signal, i.e. for example a video image, could be attached next to the imaging unit 16. The displayed video image thus gives feedback on the position of the imaging unit 16 with respect to the second subject 14 seated in the chair 10. To also give feedback on whether the vital sign detection is working correctly, a color coding, for example superimposed on the video image as a semi-transparent layer, could be used to segment the video. These segments may indicate skin parts of the first subject 12 and the second subject 14.

The identification of which skin belongs to which subject is based on the previously described methods. The skin of the second subject 14, for example, could always be indicated as light yellow and the skin of the first subject 12 as light green. The light yellow segments thus highlight skin, which has a signal matching the signal derived by the sensing unit 18 (e.g. capacitive ECG). The light green segment indicates skin with a clear pulsatile signal, which does not match the signal from the sensing unit 18, at least not for an extended period of time. However, a moderate match may exist in some cases for a short period of time. Skin or other video image parts with some pulsatile component but with an SNR below a certain threshold could be highlighted with a light grey color.

The above described yellow and green semi-transparent highlights of the video image are only present when the system 10 detects signals from the first subject 12 and the second subject 14. The yellow and green coding indicating a measurement is currently working properly may be emphasized by indicator lights e.g. green and yellow LEDs to communicate the status of the system 10 to the user, i.e. the second subject 14 or any other third person. With this visual feedback, which might be supported by an additional acoustic feedback, the measurement position of the imaging unit 16 can be adjusted until both signals are measured properly. The imaging unit 16 can be adjusted automatically or by hand.

In theory, a video image that only records the imaging signal of the first subject 12, wherein no skin of the second subject 14 is present in the image, can also provide satisfactory vital signs monitoring.

Nevertheless, the second subject 14 could also be encouraged to position the imaging unit 16 or him/herself such that skin areas of both subjects (12, 14) are measured by the imaging unit 16 and a yellow and a green segment is displayed. Thus, two vital signs are measured since this gives not only information regarding the vital sign of the second subject 14 but also helps confirming that the skin with the different value for the vital sign (different meaning not correlated with the vital sign signal of the second subject 14) is indeed that of the first subject 12.

Although in FIG. 1 the sensing unit 18 and the imaging unit 16 are connected directly to the analyzing unit 20, it is also possible that they are connected wirelessly and communicate with each other by Wi-Fi connection, Bluetooth, NFC, infrared or other techniques known in the art for transmitting data wirelessly. In this case the imaging unit 16 and the sensing unit 18 have to comprise means for sending/transmitting the obtained signals to the analyzing unit 20.

Although in the following and foregoing only a first subject 12 and a second subject 14 are discussed, it is in principle possible to monitor vital signs of more subjects, which may be helpful in the case when doing kangaroo care with twins. Therefore, it is possible to define two regions of interest, a first on the first twin and the second on the second twin and obtain signals from these regions by the same imaging unit 16, and derive vital signs from each of the twins by means of an analyzing unit 20. The system 10 may also comprise a second or even third imaging unit 16 to monitor one or more subjects from different angles, thus providing larger unexposed skin areas to one of the imaging units 16.

FIG. 3 shows a diagram, which illustrates the method according to the present invention. The method comprises obtaining (S1) the imaging signal, which is related to a first vital sign of the first subject 12 via an imaging unit 16, obtaining (S2) the sensor signal, which is related to a second vital sign of the second subject 14 from the sensing unit 18 and deriving (S3) the first vital sign of the first subject 12 from said imaging signal by taking into account said sensor signal by applying the algorithms and methods described above. Obtaining (S1) an imaging signal and obtaining (S2) a sensor signal may be carried out in parallel. But these signals may also be recorded sequentially and analyzed afterwards, in cases where a long time monitoring is sufficient and no immediate alarm is needed.

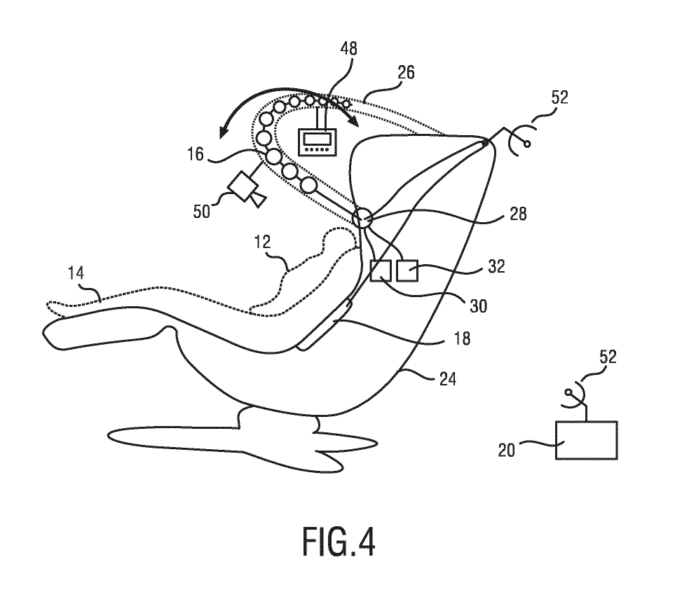

FIG. 4 shows another embodiment of the present invention in form of a so-called kangaroo care chair, wherein the system 10 for monitoring a vital sign of the first subject 12, which is in proximity or in contact with the second subject 14, is integrated into the kangaroo care chair 24. A kangaroo chair is a chair, which is optimized to support a parent or a caregiver when providing kangaroo care to a neonate or a baby.

In this embodiment the kangaroo care chair comprises a sensing unit 18, which is integrated into the chair 24. The chair 24 further comprises an arc 26 in which an imaging unit 16 for monitoring the neonate 12 from a defined distance is integrated. The arc 26 is mounted rotatably at the kangaroo chair 24 and may be rotated by means of an actuator 28. Said actuator 28 may comprise a rotary motor, a pneumatic spring or any actuator known in the art suitable for rotating or moving the arc 26.

The kangaroo care chair 24 further comprises a controller 30 for controlling the actuator 28. The arc 26 can also be arranged in an automated matter. Therefore, the kangaroo care chair comprises a controller 32 for automatically controlling the actuator 28.

The kangaroo care chair can further comprise a projector 50 for projecting light on the first subject 12, wherein the light displays the region of interest from which a camera 16, which can be integrated into the arc 26, obtains the imaging signal from the first subject 12. The projector might be arranged at the arc 26 or might be integrated into the arc 26. It is also possible to integrate an illumination unit 36 in the arc 26 and use said illumination unit 36 in order to highlight the region of interest on the first subject 12. This can be obtained by increasing the intensity of the illumination for a short time.

The kangaroo care chair 24 may further comprise a display 48 in order to display vital signs and/or other information of the measurement, like for example the region of interest from which the imaging signal is derived from the first subject 12. The kangaroo care chair 24 further comprises a transmitter 52 for transmitting the derived sensor signal and imaging signal to an analyzing unit 20, wherein the analyzing unit 20 comprises also a transceiver for receiving said signals and/or sending the derived vital signs and/or other information of the measurement. The analyzing unit 20 is configured to obtain the vital sign of the first subject 12 in the manner disclosed above.

In an alternative embodiment, the imaging unit 16, the illumination unit 36 and the display 48 are not mounted on an arc but on a flexible articulated arm 26, which can be easily flexed to optimize angle and distance to the first subject 12 and second subject 14. This flexing may be done by the second subject 14 or by a third person like caregiving staff or a visitor of the neonate.

In another embodiment of the present invention there is provided a retrofitting kit, which can be arranged at a conventional kangaroo care chair in order to enable a monitoring of a vital sign of a neonate 12 during kangaroo care in said kangaroo care chair. The retrofitting kit comprises a cover for the kangaroo care chair, wherein said cover comprises an obtrusive sensor 18 for obtaining the sensor signal. The retrofitting kit further comprises an imaging unit 16 for obtaining the imaging signal and a holding structure 26, which can be arranged at the kangaroo care chair for holding the imaging unit 16 and aligning the imaging unit 16 such that a region of interest of the neonate 12 can be observed. The retrofitting kit further comprises an analyzing unit 20, which is configured to receive the signals obtained by the sensing unit 18 and the imaging unit 16. The analyzing unit 20 is further configured to obtain the vital sign of the neonate 12 in the manner disclosed above.

Most of the smartphones available nowadays comprise transceivers for communicating with devices and a processor, which can be used to carry out the steps as described above. Consequently, such a smartphone can be used as analyzing unit 20 as well.

In yet another embodiment there is provided a sensor unit 18, like for example a contact PPG device, which can be arranged at a finger or an ear of the second subject 14. The PPG device further comprises an energy source and a transceiver for communicating with a device like, for example, a smartphone or laptop or a tablet computer. In General, every device comprising at least one camera suitable for PPG, transmitters for communicating and a processor for carrying out the steps as disclosed above can be used. In this embodiment there can also be provided a holding unit 26 for holding the device and arranging the device such that at least one camera of the device can obtain an imaging signal from the first subject 12. Thus, a camera of the device is used as imaging unit 16. Preferably, the display of the device can be used to display information of the running measurement to the second subject 14. The method for deriving a vital sign of the first subject 12 can be implemented, for example, by means of an app, which can be carried out on a smartphone or a computer program, which can be carried out on a laptop. This way it is possible to monitor the vital signs of a baby in a very comfortable way at home with low effort and costs. This embodiment provides a simple and low cost monitoring solution for the private sector.

A combination of two smartphones or other mobile electronic devices such as tablets may be used as well. In this embodiment, the camera of a first smartphone is arranged such that only the second subject 14 is monitored, thus serving as said sensing unit 18 for providing a sensor signal. The second smartphone may be placed such as to record predominantly the first subject 12 with a camera of said second smartphone, thus serving as imaging unit 16. Skin segmentation for the images derived by the second smartphone can be done by using the sensor signal derived by the first smartphone and by applying the methods disclosed above. The first and the second smartphone may communicate with an external device for performing the image and signal processing. Alternatively, the first and second smartphone could communicate with each other, wherein the first and/or the second smartphone performs the analyzing and signal processing of the derived signals. This embodiment can be implemented by means of an app, which may be installed on the first and the second smartphone.

During kangaroo care it is very challenging to distinguish the first subject 12 from the second subject 14, since a skin-to-skin contact is desired. However, by the present invention it is possible to distinguish the first subject 12 from the second subject even when they are in contact. In other words a so-called “skin separation” is achieved.

During kangaroo care the skin of the neonate and the parent will often be in contact or adjacent. This can be a challenge for a vital signs camera, which requires to have a good segmentation of the subject's skin for an accurate measurement. Algorithms to segment skin work well in normal circumstances using the significant difference between the neonate and parent heart rate as an indicator. However, in situations of reduced neonate heart rate such as a bradycardia the difference between the heart reduces considerably and can lead to incorrect skin segmentation and hence to inaccurate measurements of the vital signs.

In order to prevent that the parent's skin is processed as neonatal skin by the camera monitoring system, unobtrusive sensors (sensor that do not need skin contact, e.g. capacitive ECG/heart rate sensors, magnetic induction, pressure, sound) are integrated in the chair (e.g. embedded in the back of the chair) in order to measure the parent's vital signs.

This information is next used to separate the neonate's vital signs from the parent's, possibly by using (a combination of) signal features such as heart rate, heart rate variability in the case the heart rate of the parent and infant are the same temporarily. As an alternative to unobtrusive sensors the use of the traditional skin contact sensors, e.g. pulse oximeter probe, could be used to acquire the signal from the parent.

Although neonates or children are discussed exemplarily, the disclosed invention may be applied also for patients in general, which can benefit from kangaroo care in the sense of “being held by another person”, like for example, Alzheimer patients, severely wounded patients, who cannot bear contact measurements and still need to be monitored and wherein rPPG monitoring would be hindered by a visitor holding this person. The visitor is healthy and can easily stand a contact sensor without burden. In this case, skin segmentation can be needed even more urgently since the imaging signals may be biased even more often than during kangaroo care with neonates. While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A system for monitoring a vital sign of a first subject, which is in proximity or in contact with a second subject, the system comprising:
an imaging unit for monitoring the first subject from a distance, to obtain an imaging signal related to a first vital sign of the first subject,
a sensing unit for obtaining a sensor signal related to a second vital sign of the second subject, said sensing unit being arranged in proximity or at the second subject, wherein the first vital sign is of the same type as the second vital sign, and
an analyzing unit for deriving the first vital sign of the first subject from said imaging signal by taking into account said sensor signal and separating the first vital sign of the first subject from influences from the second vital sign of the second subject on the imaging signal.

2. The system of claim 1, wherein the imaging unit is arranged at a subject support structure or at a predetermined location with respect to the subject support structure for supporting the second subject.

3. The system of claim 1, further comprising the subject support structure, wherein the imaging unit is arranged at a holding structure, which is movably arranged at the subject support structure.

4. The system of claim 3, further comprising an actuator for actuating the holding structure and a controller for controlling the actuator based on control signals from a user.

5. The system of claim 4, further comprising a controller for automatically controlling the actuator.

6. The system of claim 1, further comprising an illumination source for illuminating the first subject.

7. The system of claim 1, wherein the sensing unit is adapted for obtaining one or more of a heart rate signal, respiratory rate signal or blood oxygen saturation signal.

8. The system of claim 1, wherein the imaging unit is adapted for obtaining photo-plethysmography, PPG, signals and wherein the analyzing unit is adapted for deriving the first vital sign of the first subject from said PPG signal by taking into account said sensor signal.

9. The system of claim 1, further comprising a display for displaying vital signs and/or other information of the measurement, wherein the region from which the PPG signal is derived is displayed by the display.

10. The system of claim 1, further comprising a projector for projecting light on a region of interest from which the PPG signal is derived.

11. The system of claim 1, wherein the analyzing unit is configured for distinguishing the skin of the first subject from the skin of the second subject in the imaging signal by breaking up the imaging signal in a plurality of non-overlapping spatial segments and correlating said segments with the second vital sign derived from the sensor signal.

12. A processor for deriving a vital sign of a first subject which is in proximity or in contact with a second subject, the processor comprising:
a first receiver for receiving an imaging signal related to a vital sign of the first subject,
a second receiver for receiving a sensor signal related to a second vital sign of the second subject, wherein the first vital sign is of the same type as the second vital sign, and
a processing unit for deriving the first vital sign of the first subject from said imaging signal by taking into account said sensor signal and separating the first vital sign of the first subject from influences from the second vital sign of the second subject on the imaging signal.

13. A method for monitoring vital signs of a first subject, which is in proximity or in contact with a second subject, the method comprising the steps of:
obtaining an imaging signal related to a first vital sign of the first subject,
obtaining a sensor signal related to a second vital sign of the second subject, wherein said sensor signal is obtained in proximity or at the second subject, wherein the first vital sign is of the same type as the second vital sign, and
deriving the first vital sign of the first subject from said imaging signal by taking into account said sensor signal and separating the first vital sign of the first subject from influences from the second vital sign of the second subject on the imaging signal.

14. A computer program comprising program code means for causing a computer to carry out the steps of the method as claimed in claim 13 when said computer program is carried out on the computer.

15. A kangaroo care chair for monitoring a vital sign of a first subject, which is in proximity or in contact with a second subject, the kangaroo care chair comprising:
an imaging unit for monitoring the first subject from a distance for obtaining an imaging signal related to a first vital sign of the first subject,
a sensing unit for obtaining a sensor signal related to a second vital sign of the second subject, said sensing unit being arranged at the kangaroo care chair, and wherein the first vital sign is of the same type as the second vital sign,
a transmitter for transmitting said imaging signal and sensing signal to an analyzing unit for deriving the first vital sign of the first subject from said imaging signal by taking into account said sensor signal and separating the first vital sign of the first subject from influences from the second vital sign of the second subject on the imaging signal.
